(19) United States
(12) Patent Application Publication
(10) Pub. No.: US 2026/0158217 A1
(43) Pub. Date: Jun. 11, 2026

(54) DIABETES MANAGEMENT SYSTEM

(71) Applicant: Imam Abdulrahman Bin Faisal University, Dammam (SA)

(72) Inventors: Gadah Abdulrahman BAAQEEL, Dammam (SA); Dana ALHUSSAIN, Dammam (SA); Ruba ALKHALDI, Dammam (SA); Zati HAZIRAT, Dammam (SA); Najla Ibrahim ALRUMAIHI, Dammam (SA)

(73) Assignee: Imam Abdulrahman Bin Faisal University, Dammam (SA)

(21) Appl. No.: 18/972,059

(22) Filed: Dec. 6, 2024

(51) Int. Cl. A61M 5/172; A61M 37/00

(52) U.S. Cl. CPC A61M 5/1723; A61M 37/0015; A61M 2005/1726; A61M 2037/0023; A61M 2205/3303; A61M 2205/3569; A61M 2230/06; A61M 2230/201; A61M 2230/50

(57) ABSTRACT

A diabetes management system including a wearable device having an elongated support patch with an adhesive disposed on a skin side, a sensor portion disposed on a non-skin side and including an electrochemical glucose sensor and a wireless communication module, and a medication delivery portion disposed on a non-skin side and comprising a medication reservoir and a medication pump. The system also includes a behavior tracking device having a tracking wireless communication module, a portable electronic device configured to communicate wirelessly to the wearable device and the behavior tracking device, and a controller. The sensor portion is configured to detect a glucose level of a subject and the controller is configured to receive the glucose level and to transmit to the medication pump an instruction which causes the medication pump to deliver a dose to the subject of a medication based on the glucose level.

### BACKGROUND OF THE INVENTION

### Field of the INVENTION

The present disclosure relates to a diabetes management system, including a wearable device a behavior tracking device, a portable electronic device, and a controller.

### Discussion of the Background

The “background” description provided herein is for the purpose of generally presenting the context of the disclosure. Work of the presently named inventors, to the extent it is described in this background section, as well as aspects of the description which may not otherwise qualify as prior art at the time of filing, are neither expressly nor impliedly admitted as prior art against the present invention.

Diabetes has been known to be an irreversible disorder leading to increased blood glucose levels. Current type 1 diabetes treatment includes administering insulin, exercising, and making dietary changes. Current type 2 diabetes treatment includes administering insulin and/or non-insulin medications, exercising to reduce weight, or making dietary changes. Current estimates predict approximately 643 million people aged 20 to 79 will have diabetes by 2030. Globally, the cost of treating diabetes is high. For example, the average of lifetime cost of treating diabetes in the U.S.A. is $85,000.

Closed loop automated medication delivery systems, such as automatic insulin dosing systems are currently a promising solution for the control of blood glucose. The automated medical delivery systems may be informed with current blood glucose levels and trends to drive the automated medication delivery system. However, these systems are imperfect. Challenges remain because of factors such as variability in medication sensitivity, effects from ongoing exercise or recent exercise, other medications being taken by the patient, other behavioral factors such as smoking, the amount, type, and recency of food consumption, sleep, and many others. For example, glucose uptake into skeletal muscle can persist for several hours after exercise. Consequently, there is a significant risk of exercise induced hypoglycemia (low blood sugar) if the effects of the exercise are not accounted for. Further, effects from other behaviors and activities can also affect the use of insulin to treat diabetes, such as alcohol consumption, smoking, and the use of other, non-insulin medications. The amount of rest a user has had recently can have a dramatic impact on caloric need and glucose levels.

Accordingly, it is an objective of the present disclosure to provide a diabetes management system that can detect and/or account for user behaviors that can affect the treatment of the user's diabetes.

### SUMMARY OF THE INVENTION

The present disclosure relates to a diabetes management system, comprising a wearable device that includes an elongated support patch including an adhesive disposed on a skin side of the elongated support patch, wherein the elongated support patch has a first rounded end and a second rounded end, with the first rounded end smaller than the second rounded end, a sensor portion disposed on a non-skin side of the elongated support patch at the first rounded end and comprising an electrochemical glucose sensor, and a wireless communication module, and a medication delivery portion disposed on a non-skin side of the elongated support patch at the second rounded end and comprising a medication reservoir and a medication pump, a behavior tracking device comprising a tracking wireless communication module, a portable electronic device configured to communicate wirelessly to the wearable device and the behavior tracking device, and a controller, wherein the sensor portion is configured to detect a glucose level of a subject and the controller is configured to receive the glucose level and to transmit to the medication pump an instruction which causes the medication pump to deliver a dose to the subject of a medication based on the glucose level.

In some embodiments, the elongated support patch is formed from a polymer film.

In some embodiments, the elongated support patch is formed from a textile.

In some embodiments, the elongated support patch has a maximum thickness of 0.25 to 5 mm.

In some embodiments, the sensor portion further comprises a heartbeat sensor and a temperature sensor, the controller is further configured to receive a heartbeat level and a temperature of the subject, and the instruction is further based on the heartbeat level and the temperature of the subject.

In some embodiments, the sensor portion has a maximum thickness of 1 to 8 mm.

In some embodiments, the medication delivery portion further comprises a microneedle.

In some embodiments, the medication reservoir has a medication solution capacity of 1 to 5 mL.

In some embodiments, the medication delivery portion has a wedge-shaped cross section having a maximum thickness of 5 to 15 mm.

In some embodiments, the medication in insulin.

In some embodiments, the controller is included in the sensor portion of the wearable device.

In some embodiments, the controller is included in the portable electronic device.

In some embodiments, the behavior tracking device is configured to track a user behavior associated with the medication delivered by the system.

In some embodiments, the user behavior is associated with a change in a dosage of the medication delivered by the system.

In some embodiments, the behavior tracking device is configured to transmit a behavior signal to the controller, the controller is configured to receive the behavior signal, and the instruction is further based on the behavior signal.

In some embodiments, the portable electronic device is configured to provide a user notification based on the user behavior associated with the medication delivered by the system.

The present application also relates to a method of managing diabetes in a subject, the method comprising detecting a glucose level of the subject using the sensor portion of the diabetes management system and delivering to the subject a dose of a medication based on the glucose level, the delivering being performed by the diabetes management system.

In some embodiments, the method further includes detecting a user behavior associated with the medication delivered by the diabetes management system, wherein the dose of the medication is based on the user behavior.

In some embodiments, the method further includes providing a user notification based on the user behavior associated with the medication delivered by the system, the user notification being provided by the portable electronic device of the diabetes management system.

In some embodiments, the medication is insulin.

### BRIEF DESCRIPTION OF THE DRAWINGS

FIG. 1A shows a top-down schematic view of an exemplary embodiment of a wearable device according to the present specification;

FIG. 1B shows a top-down schematic view of an exemplary embodiment of a wearable device according to the present specification;

FIG. 1C shows a side-on schematic view of an exemplary embodiment of a wearable device according to the present specification;

FIG. 1D shows a side-on schematic view of an exemplary embodiment of a wearable device according to the present specification;

FIG. 1E shows a frontal schematic view of an exemplary embodiment of a wearable device according to the present specification;

FIG. 1F shows a frontal schematic view of an exemplary embodiment of a wearable device according to the present specification;

FIG. 1G shows a three-quarters schematic view of an exemplary embodiment of a wearable device according to the present specification;

FIG. 1H shows a three-quarters schematic view of an exemplary embodiment of a wearable device according to the present specification;

FIGS. 2A-2D show renderings of an exemplary embodiment of a wearable device according to the present specification with FIGS. 2A-2C showing different three-quarters views and FIG. 2D showing a side-on view;

FIG. 3 shows an expanded view of an exemplary embodiment of a wearable device according to the present specification;

FIG. 4 shows a rendering showing an exemplary embodiment of a behavior tracking device according to the present specification; and

FIGS. 5A-5C show exemplary depictions of performing a method of diabetes management using the system according to the present specification with FIGS. 5A-5B showing a subject wearing the wearable device and FIG. 5C showing the behavior tracking device placed on a medication bottle.

### DETAILED DESCRIPTION OF THE INVENTION

In the following description, it is understood that other embodiments may be utilized and structural and operational changes may be made without departure from the scope of the present embodiments disclosed herein.

### Definitions

As used herein the words “a” and “an” and the like carry the meaning of “one or more.”

As used herein, the terms “optional” or “optionally” means that the subsequently described event(s) can or cannot occur or the subsequently described component(s) may or may not be present (e.g., 0 wt. %).

According to a first aspect, the present disclosure relates to a diabetes management system. The system includes a wearable device, a behavior tracking device, a portable electronic device, and a controller.

### Wearable Device

The wearable device includes an elongated support patch. In some embodiments, the elongated support patch has a first rounded end and a second rounded end. In some embodiments, the first rounded end is smaller than the second rounded end. The elongated support patch can be a substantially flattened shape having a length, a width, and a thickness. In some embodiments, the elongated support patch has a length of 50 to 100 mm, preferably 60 to 97.5 mm, preferably 65 to 95 mm, preferably 70 to 92.5 mm, preferably 75 to 90 mm, preferably 77.5 to 87.5 mm, preferably 80 to 85 mm. In some embodiments, the elongated support patch has a maximum width of 15 to 40 mm, preferably 17.5 to 35 mm, preferably 20 to 30 mm, preferably 22.5 to 27.5 mm. In preferred embodiments, the maximum width of the elongated support patch is defined by the second rounded end. In some embodiments, the elongated support patch has a maximum thickness of 0.25 to 5 mm, preferably 0.5 to 3 mm, preferably 0.75 to 2.5 mm, preferably 1 to 2 mm.

In some embodiments, the elongated support patch has a middle portion formed between the first rounded end and the second rounded end. In some embodiments, the middle portion has a width which is equal to or smaller than a width of the second rounded end. In some embodiments, the middle portion has a width which is equal to or smaller than a width of the first rounded end. In some embodiments, the elongated support patch has an asymmetric “bone” shape or “dumbbell” shape. A dumbbell shape is one that generally has rounded ends separated by a narrow central portion with a width less than that of the rounded ends. In general, the first rounded end and second rounded end may have a shape similar to that of a flattened circle, ellipsoid, ovoid, or a shape similar to another object which may be described as rounded. The middle portion may have straight sides or curving sides. A portion of the elongated support patch where the rounded ends meet the middle central portion may have a tapered shape or other gradual change in width from that of the rounded ends to that of the middle portion.

In some embodiments, the elongated support patch is configured to be flexible. In some embodiments, the elongated support patch is configured to be flexible along an axis connecting the first rounded end to the second rounded end. That is, the elongated support patch is flexible along its length. Such flexibility can be or permit, for example, bending of the rounded ends toward each other. In some embodiments, the elongated support patch is configured to be flexible along an axis perpendicular to an axis connecting the first rounded end to the second rounded end. That is, the elongated support patch is flexible along its width. Such flexibility can be or permit a twisting of the elongated support patch along an axis connecting the first rounded end to the second rounded end. Such flexibility can ensure that the elongated support patch can conform to the body surfaces of the user both when the user is still and when the user moves. This flexibility may also provide a distinct advantage associated with placing the sensor portion and medication delivery portion at opposite ends of the elongated support patch. Such a separation, combined with the flexibility of the elongated support patch can impart onto the wearable device enhanced benefit in terms of permitting the user free motion or action while remaining secure on the user and/or by permitting a smaller total size. These advantages may also be associated with giving the wearable device an overall size and/or shape better suited for dressing/undressing, performing daily activities, exercising, or other movement- or motion-intensive activities while wearing the device. Such activities can be performed uninhibited by an inflexible or bulky device. Further, the wearable device can conform to local changes in the shape of the user's body associated with motion, such as skin stretching or compression, muscle contraction, or changes in orientation with respect to gravity.

In some embodiments, the flattened shape of the elongated support patch defines a skin side and a non-skin side. In preferred embodiments, a skin side of the elongated support patch is substantially flat. That is, the skin side of the elongated support patch has a deviation of thickness of less than 10%, preferably less than 7.5%, preferably less than 5% of an average thickness of the skin side. In some embodiments, the skin side is configured to be in contact with a subject's skin. In some embodiments, the non-skin side is configured to have other components disposed it so as to attach those components to the elongated support patch. Examples of such other components include, but are not limited to the sensor portion, wireless communication module, and medication delivery portion described below.

In some embodiments, the elongated support patch includes an adhesive disposed on a skin side of the elongated support patch. In general, the elongated support patch can include any adhesive that provides suitable adhesion to the skin and is suitable for use on the skin (e.g., the adhesive should preferably be non-irritating and non-sensitive). Examples of particularly suitable adhesives are pressure sensitive adhesives. In some embodiments, the pressure sensitive adhesive has a relatively high moisture vapor transmission rate to allow for evaporation of moisture. Examples of suitable pressure sensitive adhesives include acrylate-based, polyurethane, hydrogel, hydrocolloid, block copolymer, silicone, rubber-based adhesives (including natural rubber, polyisoprene, polyisobutylene, butyl rubber, and the like), and combinations of these adhesives. The adhesive composition may include tackifiers, plasticizers, rheology modifiers, and active ingredients including, for example, antimicrobials.

Examples of pressure sensitive adhesives that may be used may include adhesives commonly applied to the skin such as acrylate copolymers as described in U.S. Pat. No. RE24,906, particularly 97:3 isooctylacrylate acrylamide copolymers. Another example may include a 70:15:15 terpolymer of isooctyl acrylate ethylene oxide acrylate acrylic acid, as described in U.S. Pat. No. 4,737,410, each of which is incorporated herein by reference in its entirety. Other potentially useful adhesives are described in U.S. Pat. Nos. 3,389,827, 4,112,213, 4,310,509 and 4,323,557, each of which is incorporated herein by reference in its entirety. In some embodiments, a pharmaceutical or antimicrobial agent is included in a binder as described in U.S. Pat. Nos. 4,310,509 and 4,323,557, each of which is incorporated herein by reference in its entirety. Silicone adhesives may also be used. Generally, silicone adhesives can provide suitable adhesion to skin while being capable of being gently removed from the skin. Examples of suitable silicone adhesives are disclosed in PCT publications WO2010/056541 and WO2010/056543, each of which is incorporated herein by reference in its entirety.

In some embodiments, the pressure sensitive adhesive can transmit moisture at a rate greater than or equal to the rate at which human skin transmits moisture. While such properties can be achieved by selecting an appropriate adhesive, it is also contemplated in the present invention that other methods of achieving a high relative rate of moisture transmission can be used, such as a mode of applying the adhesive to a backing, as described in U.S. Pat. No. 4,595,001, which is incorporated herein by reference in its entirety. Other potentially suitable pressure sensitive adhesives may include Blown Microfiber (BMF) adhesives such as those described in U.S. Pat. No. 6,994,904. The pressure sensitive adhesive used may also include one or more regions where the adhesive itself includes a structure such as a microreplicated structure as described in U.S. Pat. No. 6,893,655, which is incorporated herein by reference in its entirety.

In some embodiments, the elongated support patch is formed from a polymer film. Examples of suitable polymers which may be used to form the polymer film include, but are not limited to polyurethane, polyethylene, vinylester, epoxys, polyamides, polypropylene, polyethylene terephthalate, polybutylene terephthalate, polyetherimide, polyvinylidene fluoride, polyetheretherketone, poly(acrylonitrile butadiene styrene) (ABS), polylactic acid (PLA), polycarbonate, polyvinyl chloride silicones, and any permissible combinations or copyolymers thereof. Preferably, the polymer film is flexible and stretchable to conform to the user's body.

In some embodiments, the elongated support patch is formed from a textile. In general, the textile can be formed from any suitable fibers. Examples of suitable fibers for forming the textile include, but are not limited to polyester, nylon, polyurethane, Lycra® or other synthetic or natural fibers. In some embodiments, the textile elastomeric properties that come from properties of the fibers themselves, or from how the fibers are combined to form the textile. The textile can be woven, non-woven, knitted, spun or constructed of a textured film. In some embodiments, the textile, or portions thereof can be conductive. Conductive aspects of the textile can come from fine metal wires, either in the yarn used to make the textile or woven into the textile alongside non-conductive fibers. The electrical properties of textile can come from inherently conductive polymers or nanocomposites deposited as coatings on the fabric's fibers.

Various components of the wearable device can be woven directly into the textile of the elongated support patch, including, but not limited to, complex electronic pathways, circuits, controls, electrodes, sensors, traces, connectors, resistors, antenna, batteries, switches and other components. Switches, buttons, and other controls can be incorporated into the textile by using a multilayered fabric. For example, three electro-active layers can be used. Two outer conductive layers can surround an inner resistive layer that separates the conductive layers until the layers are momentarily pressed together.

Using a textile as discussed above may be associated with advantageous properties or qualities of the elongated support patch. For example, the elongated support can be soft, stretchable and breathable to provide user comfort during use. Textile supports can provide a high moisture vapor transmission rate (MVTR). Textiles can be rolled, crumpled and folded without damaging functionality.

In some embodiments, the elongated support path may also be constructed or coated to be flame resistant, waterproof or water-resistant.

The wearable device also includes a sensor portion disposed on a non-skin side of the elongated support patch at the first rounded end. In some embodiments, the sensor portion comprises a glucose sensor. In general, the glucose sensor can be any suitable glucose sensor known to one of ordinary skill in the art. Typically, glucose sensors are classified as magnetic, optical, piezoelectric, thermo-responsive, or electrochemical based on the reporting principles used in their operation. Glucose sensors can operate on molecular recognition elements including various glucose-sensitive enzymes, receptors, nucleic acids, antibodies, microorganisms, and lectins. In some embodiments, the glucose sensor is an electrochemical glucose sensor. Electrochemical glucose sensors may be advantageous for increases in factors such as sensitivity, reliability, reproducibility, shelf-life stability, convenient maintenance, and/or ease of calibration. Typically, electrochemical glucose sensors are divided into three types: potentiometric, amperometric, or conductometric sensors. In general, any of the types of electrochemical glucose sensors may be used.

In some embodiments, the glucose level can be a blood glucose level. In some embodiments, the glucose level can be an interstitial glucose level. Blood glucose levels can be estimated (e.g., by the glucose sensor, the controller, the portable electronic device, or some other device) based on the measured signals indicative of the interstitial glucose levels. A glucose sensor may be inserted directly into a blood vessel or require blood samples (e.g., drawn from a finger prick) to measure blood glucose (BG) levels directly. Such a sensor can be referred to as a “blood glucose sensor” or other similar term indicating that the sensor measures blood. A glucose sensor may be inserted into subcutaneous tissue to measure electrical signals indicative of the glucose level in the interstitial fluid. Based on the electrical signals indicative of the interstitial glucose levels, blood glucose levels can be indirectly estimated (e.g., by converting the interstitial glucose measurements into blood glucose estimates based on a calibration process). These estimated blood glucose measurements may sometimes be referred to as sensor glucose (SG) data/values. Although SG values may not exactly match BG measurements taken directly from blood, SG can be a highly representative and accurate estimation of BG.

In some embodiments, the glucose sensor can be configured to substantially continuously (e.g., every 5 min, every 15 minutes, every hour, etc.) measure signals indicative of a glucose level of a user.

In some embodiments, the sensor portion may include other types of sensors that may be worn, carried, or coupled to a user to measure user activity that may influence the glucose levels or glycemic response of the user. As an example, such sensors may include an acceleration sensor configured to detect an acceleration of the user or a portion of the user, such as hands, arms, legs, or feet. The acceleration (or lack thereof) may be indicative of exercise, sleep, or food/beverage consumption activity of the user, which may influence the user's glycemic response, glucose need, and/or medication need. In some embodiments, the sensors may include a heartbeat (heart rate) sensor. In some embodiments, the sensor portion may include a temperature sensor, such as a body temperature sensor. Such sensors may be useful for indicating an amount of physical exertion performed by a user as described below. In some embodiments, the sensors may include a GPS receiver which detects GPS signals to determine a location, movement, and/or location history of the user.

The sensors described above are merely provided as examples. Other sensors or types of sensors for monitoring physiological condition, activity, and/or location, among other things, will be recognized by persons skilled in the art and are contemplated to be within the scope of the present disclosure.

The sensor portion is configured to detect is configured to detect a glucose level of a subject (e.g., a user) as described above. In some embodiments, the sensor portion is configured to transmit to the controller the glucose level of a subject (user). In some embodiments, the sensor portion is further configured to detect an activity or activity level of a user. As the user's activity can impact a current or future glucose level, glucose need, or medication need of a user, detection of the user's activity level (e.g., detection of activity and/or specific parameters of an activity) can be advantageous for determining the current and/or future medication need of the user. For example, the sensor portion can include a heartbeat sensor and a temperature sensor as described above. Such a sensor portion can detect the heart rate and body temperature of the user. Such heart rate and body temperature data can be transmitted to the controller. The controller can then cause the medication delivery portion (described below) to deliver a certain amount of medication (e.g., insulin) to a subject (user) based on the detected heart rate, body temperature, or other parameter. The controller can also adjust a diabetes treatment plan, medication regimen, dosing schedule, or program. For example, the controller can cause the medication delivery portion to deliver an additional dose, skip a dose, deliver a larger or smaller dose, postpone or expedite a scheduled dose, or otherwise change a medication delivery parameter or schedule in response to the user's activity, such as performing exercise or having a meal as described below.

In some embodiments, the sensor portion has a maximum thickness of 1 to 8 mm, preferably 1.5 to 7 mm, preferably 2 to 6 mm, preferably 2.5 to 5.5 mm, preferably 3 to 5 mm, preferably 3.25 to 4.75 mm, preferably 3.5 to 4.5 mm, preferably 3.75 to 4.25 mm, preferably 4 mm.

In some embodiments, the sensor portion comprises a wireless communication module. The wireless communication module can enable or facilitate transmission of electronic information between the sensor portion and some other component of the system, such as the controller, the medication delivery portion, the behavior tracking device, the portable electronic device, combination of these, or other suitable device or component. In general, the wireless communication module can use any protocol or method for transmitting electronic information, such as Wifi, Bluetooth®, AirPlay®, EDGE, wireless cellular systems such as 3G, 4G and 5G, and the like.

The wearable device also includes a medication delivery portion disposed on a non-skin side of the elongated support patch at the second rounded end. In some embodiments, the medication delivery portion comprises a medication reservoir and a medication pump. In some embodiments, the medication delivery portion is configured to deliver to a subject (user) a medication. In some embodiments, the medication is a liquid medication. In some embodiments, the liquid medication comprises insulin. In some embodiments, the medication is insulin. In some embodiments, the insulin is in the form of an insulin solution.

In some embodiments, the medication delivery portion comprises a microneedle. In some embodiments, the microneedle is configured to be placed into a blood vessel (e.g., an artery, a vein, or a capillary) of a subject (user). In some embodiments, the microneedle is configured to be placed into a tissue of a subject (user). In some embodiments, the microneedle is configured to deliver the medication by transdermal delivery. In some embodiments, the microneedle is configured to deliver the medication by injection. In some embodiments, the injection is subcutaneous injection. In some embodiments, the injection is intravenous injection. In some embodiments, the medication delivery portion comprises a single microneedle. In some embodiments, the medication delivery portion comprises a plurality of microneedles. In some embodiments, the plurality of microneedles is arranged to form a microneedle array.

In some embodiments, the microneedle is disposed at a location such that when the microneedle is placed in a blood vessel or into a tissue of the subject, the microneedle itself is covered by the support patch or a portion thereof. For example, the location where the microneedle enters the subject's skin can be covered by a portion of the support patch, such as the second rounded end. In some embodiments, the microneedle is disposed at a location such that when the microneedle is placed in a blood vessel or into a tissue of the subject, the microneedle itself is not covered by the support patch or a portion thereof. That is, the microneedle can be placed in the subject at a location remote to the support patch. In such an embodiment, the microneedle can be connected to other components of the wearable device, e.g., the medication reservoir, the medication pump, etc. by a tube, cannula, or other similar implement that can allow fluid transfer from the other components of the wearable device to the microneedle for medication delivery.

The medication reservoir may be configured to store an amount of the medication. In some embodiments, the medication reservoir may be refillable or replaceable. In some embodiments, the medication reservoir can have a refill opening or refill port configured to allow a person to fill the medication reservoir with the medication. In some embodiments, the medication reservoir can be detachable from other portions of the medication delivery device. For example, the medication reservoir can have a medication exit port through which the medication is configured to pass during a delivery action. The medication exit port can align, interface with, or otherwise interact with an appropriate portion or structure of the medication pump.

In some embodiments, the medication reservoir (reservoir) has a medication solution capacity of 1 to 5 mL, preferably 1.25 to 4.75 mL, preferably 1.5 to 4.5 mL, preferably 1.75 to 4.25 mL, preferably 2.0 to 4.0 mL, preferably 2.1 to 3.9 mL, preferably 2.2 to 3.8 mL, preferably 2.3 to 3.7 mL, preferably 2.4 to 3.6 mL, preferably 2.5 to 3.5 mL, preferably 2.6 to 3.4 mL, preferably 2.7 to 3.3 mL, preferably 2.8 to 3.2 mL, preferably 2.9 to 3.1 mL, preferably about 3.0 mL. In some embodiments, the medication delivery portion has a wedge-shaped cross section having a maximum thickness of 5 to 15 mm, preferably 5.5 to 14 mm, preferably 6.0 to 13 mm, preferably 6.5 to 12 mm, preferably 7.0 to 11 mm, preferably 7.25 to 10 mm, preferably 7.5 to 9.0 mm, preferably 7.75 to 8.5 mm, preferably 8.0 mm. In some embodiments, the wedge-shaped cross section has rounded edges. In some embodiments, the wedge-shaped cross section is devoid of sharp corners. In some embodiments, the wedge-shaped cross section is a cross-section in a direction perpendicular to a plane defined by the support patch. In some embodiments, the medication reservoir has an elliptical cross-section in a direction substantially parallel to a plane defined by the support patch. It should be understood that the term “elliptical cross-section” includes a circular cross-section. In some embodiments, the wedge shape advances from a low height edge to a high height edge at an angle of 5 to 45°, preferably 10 to 30° or about 15 to 25° from the plane of the support.

In general, the medication pump can be any suitable pump known to one of ordinary skill in the art. Examples of pumps include, but are not limited to, shape memory alloy actuator pumps (also called “nitinol wire pumps”), step motor pumps, piezoelectric pumps, spring-driven pumps, bladder pumps, ultrasonic pumps, and the like. The medication pump is preferably configured to facilitate delivery of an aliquot of medication to the user. In some embodiments, the medication pump is configured to deliver the aliquot in response to a signal from the controller. In some embodiments, the aliquot can be referred to as a “bolus”.

In some embodiments, the wearable device comprises a user input device. The user input device can be any suitable such device known to one of ordinary skill in the art. The user input device can include a screen. For example, the electronic user input device can be a keypad and/or touchscreen. The user input device is configured to receive user input and provide instructions to the controller based on the user inputs. Such user inputs can be, include, or correspond to any action related to delivery of the medication. For example, the user input device can be used to receive user inputs that cause the medication delivery portion to deliver a dose to the subject of the medication. Such user input can be transmitted to the controller, which transmits the medication pump an instruction which causes the medication pump to deliver the dose.

In some embodiments, the wearable device can include a power source. Examples of power sources include, but are not limited to a battery, a capacitor, a piezoelectric device, and other forms of energy harvesting devices. The power source may be useful for supplying electrical power to the components of the wearable device, such as the sensors included in the sensor portion, the medication pump, the controller, the wireless communication module, and/or other components.

The power source may be a rechargeable battery or a number of rechargeable batteries. The wearable device may include appropriate hardware for charging the rechargeable battery/batteries. For example, the wearable device may include a charging port configured to accept a charging cable for supplying electrical power to the rechargeable battery/batteries. The cable may be any such cable such as such as USB, mini-USB or the like, a wall outlet, an automobile 12V power, a renewable power source or the like. In another example, the wearable device may include a wireless charging receiver interface. Such a wireless charging receiver can be configured to connect to or interface with an appropriate wireless charging supplier interface. Such connecting or interfacing may be accomplished with magnets, reusable adhesive, mechanical interference fit clamps, or any other method. In another example, the rechargeable battery/batteries (and/or other power source) may be configured to be removed and replaced such that the power source can be swapped by the subject (user) as needed.

In some embodiments, the wearable device includes a notification device. In general, the notification device can be any suitable device capable of alerting, notifying, or otherwise getting a subject's (user's) attention. In general, the notification device can be or include a device for generating an audible notification (e.g., a noise), a tactile notification such as a vibration, rumble, shake, or the like, a visual notification such as a light, flash, blink, color change, or the like, or a combination of these. In general, the notification device can be or include any suitable hardware for generating the aforementioned notification(s). For example, the notification device can include a vibration-inducing device. Examples of vibration-inducing devices include, but are not limited to a piezoelectric linear vibration motor (also referred to as a piezoelectric actuator), a rotary vibration motor, a linear vibration motor (also referred to as a linear resonant actuator), and a solenoid vibration motor. A rotary vibration motor can include an off-center or off-balance mass connected to a central rotating portion. Such a rotary vibration motor can be referred to as a “rotating eccentric mass vibration motor”. Linear Resonant Actuators (LRAs) are commonly found in wearables and smartphones. LRA motors can include a magnet attached to a spring, surrounded by an electromagnetic coil and housed in a casing. The coil is used to drive the motor by moving the mass back and forth within the housing, creating the vibrations. Piezoelectric actuators function by applying a voltage to piezoelectric material, which causes it to change shape and produce vibration. These actuators can produce highly detailed haptic feedback, making them advantageous for applications where precision is key, such as in medical devices. Linear Magnetic Ram (LMR) functions using solid-state magnetic suspension technology. LMR vibration motors produce vibration by driving a suspended mass through a magnetic field. This movement is controlled by an electric current, which can be adjusted to change the position, speed, and force of the mass. The notification device, for example, can be or include a light such as an LED. The LED can blink, flash, change color, or otherwise change some feature of its operation to provide the notification. The notification device, for example, can be or include a speaker. The speaker can, for example, play a specific sound to provide the notification. The speaker can, for example, play a pre-recorded or procedurally generated voice to provide the notification.

In some embodiments, the wearable device includes a cover. In some embodiments, the cover can be disposed upon the sensor portion, the medication delivery portion, or both. In some embodiments, the wearable device includes a single cover. That single cover can cover any suitable portion of the wearable device, including an entirety of the wearable device. In some embodiments, the wearable device includes more than one cover. For example, the wearable device can include two separate covers. One such cover can be disposes on the sensor portion. Another such cover can be disposed on the medication delivery portion. In general, the cover can be formed from any suitable material described above. In some embodiments, the cover is formed from a rigid material, such as metal or a rigid polymer. The rigid material may serve to or be advantageous for providing protection to the wearable device or portion thereof disposed under the cover. In some embodiments, the rigid material may be further covered by another material, such as a textile, silicone, flexible polymer, or the like. This other material may be advantageous for protecting a subject (user) or for increasing subject (user) comfort.

The components of the wearable device described above are merely provided as examples. The wearable device may include other components, such as, without limitation, a power supply, a communication transceiver, computing resources, and/or user interfaces, among other things. Persons skilled in the art will recognize various implementations of the wearable device and the components of such implementations. All such implementations and components are contemplated to be within the scope of the present disclosure.

### Behavior Tracking Device

In some embodiments, the behavior tracking device is configured to track a user behavior associated with the medication delivered by the system or with the diabetes of the subject (user). Such tracking can be useful for accounting for impacts of the behavior on the user's diabetes treatment with insulin or by other treatment methods. The tracking may also be useful for improving the effectiveness of diabetes treatment by, for example, increasing user compliance, increasing the user's performance of behaviors associated with improved diabetes treatment or management, or for decreasing the user's performance of behaviors associated with worse diabetes treatment or management.

Many persons who have diabetes use a variety of behaviors and/or non-insulin medications to manage their diabetes. These behaviors and/or non-insulin medications can have dramatic impacts on the person's glucose need, glycemic index, or insulin need. Additionally, many normal behaviors or the specifics of normal behaviors are known to have an impact on the person's glucose need, glycemic index, or insulin need. For example, the amount or quality of sleep a person gets in a night can impact their diabetes or the management thereof in the following day or days. Because of the possible impact on a person's glucose need, glycemic index, or insulin need, tracking these behaviors or the use of non-insulin medications can be particularly advantageous for determining a person's diabetes treatment plan, medication regimen, dosing schedule, or program.

The behavior tracking device is configured to track and thereby allow the system to automatically account for that behavior. For example, a subject may have a certain non-insulin medication they are prescribed or use intermittently that can impact the person's glucose need, glycemic index, or insulin need immediately after taking or for the entire day after taking. When the subject takes that certain non-insulin medication, they have a different glucose need, glycemic index, or insulin need compared to when they do not take that certain non-insulin medication. The behavior tracking device can be configured to detect when the subject takes that certain non-insulin medication (e.g., by detecting the subject opening the bottle storing the non-insulin medication). The behavior tracking device can communicate wirelessly with the controller (and/or any other suitable device) such that the diabetes management system can adjust the diabetes treatment plan, medication regimen, dosing schedule, or program accordingly as described below. This automatic detection may be particularly advantageous compared with having the subject manually enter their use of the non-insulin medication. Such advantages may be in the form of better diabetes management by some management metric. The behavior tracking device can, for example, track the amount or quality of a person's sleep, the amount or identity of a food or foods the person is consuming, a user's smoking or substance use, or other similar activity or behavior.

In some embodiments, the user behavior is associated with a change in a dosage of the medication delivered by the system. Such a change may be due to, for example, a change in the caloric expenditure, glucose need, medication need, or the like.

In some embodiments, the behavior tracking device includes a tracking wireless communication module. The wireless communication module can enable or facilitate transmission of electronic information between the sensor portion and some other component of the system, such as the controller, the medication delivery portion, the behavior tracking device, the portable electronic device, combination of these, or other suitable device or component. In general, the wireless communication module can use any protocol or method for transmitting electronic information, such as Wifi, Bluetooth®, AirPlay®, EDGE, wireless cellular systems such as 3G, 4G and 5G, and the like.

In some embodiments, the behavior tracking device is configured to transmit a behavior signal to the controller, the controller is configured to receive the behavior signal, and the instruction is further based on the behavior signal.

In some embodiments, the behavior signal can also be used to generate a notification or alert to the user. Such a notification or alert can be used to attempt to influence a behavior associated with the behavior tracking device. For example, the alert or notification can remind the user to take the non-insulin medication or to consume a specific food. The alert or notification can encourage the user to increase the frequency, duration, or other property of the behavior associated with the behavior tracking device. The alert or notification can alternatively or in addition discourage the user from engaging in the behavior associated with the behavior tracking device. For example, the alert or notification can discourage the user from smoking if it detects the user has initiated a process of smoking or discourage the user from eating the specific food associated with the behavior tracking device. Such encouragement and/or discouragement can be useful for helping the user form habits or continue to perform activities other than insulin administration or use that better manage the patient's diabetes and/or help improve the function of the diabetes management system. Such habits or activities can form part of a diabetes treatment or management plan.

### Controller

In general, the controller can serve to control various components and functions of the diabetes management system. One such function is to control the delivery of the medication by operation of the medication pump. In some embodiments, the controller is configured to receive the glucose level (e.g., from the sensor portion) and to transmit to the medication pump an instruction which causes the medication pump to deliver a dose to the subject of a medication. In some embodiments, the dose of the medication is based on the glucose level as described below.

In some embodiments, the controller is included in the sensor portion of the wearable device. In some embodiments, the controller is included in the portable electronic device. In some embodiments, the controller is included in some other device that is or forms a component of the diabetes management system. In general, the controller can communicate with (e.g., transmit signals to and/or receive signals from) any other components of the diabetes management system as appropriate. For example, the controller can communicate with a component located within the same device by an appropriate wired connection. The controller can communicate with a component not located within the same device by an appropriate wireless connection.

In some embodiments, the controller is configured to provide an instruction that causes the medication delivery portion to deliver a certain amount of medication (e.g., insulin) to a subject (user). The instruction can include an amount of medication to be delivered and/or a medication delivery rate. The instruction can include any other suitable parameter related to the delivery of medication known to one for ordinary skill of the art.

In some embodiments, the controller can initiate a medication delivery action in response to a user input. In general, that user input can be provided to the controller in any suitable way. In an example, the user can initiate a medication delivery action by interacting with a user input device integrated with the wearable device as described above. In an example, the user can initiate a delivering by providing an input to the portable electronic device (e.g., a smartphone) as described above. The portable electronic device can then transmit to the controller a signal that cause the controller to initiate the medication delivery action.

In some embodiments, the controller can initiate a medication delivery action automatically. That is, the controller can send an instruction that causes the medication pump to deliver a dose to the subject of a medication without the instruction (e.g., medication delivery action) being initiated by a user input. The instruction can be provided based on a diabetes treatment plan, medication regimen, dosing schedule, or program. Such a diabetes treatment plan, medication regimen, dosing schedule, or program can include, for example, user-specific information relating to the management of the user's diabetes such as the user's weight, resting or normal heartrate, normal medication need, normal caloric expenditure, typical medication response, and the like. Such information can be useful in determining the amount of medication to be delivered and/or the medication delivery rate. For example, the controller can, based on the regimen, cause to be delivered a single dose of the medication when a user is scheduled to take the dose. In another example, the controller can cause to be delivered one or more doses of the medication before the user is schedule to take them.

In some embodiments, the controller is configured to receive a diabetes treatment plan, medication regimen, dosing schedule, or program of the medication held in the reservoir. In some embodiments, the controller is configured to deliver the medication at a particular time based on the diabetes treatment plan, medication regimen, dosing schedule, or program. In general, the diabetes treatment plan, medication regimen, dosing schedule, or program can be input or retrieved by the controller as described above. In some embodiments, the controller is configured to receive a diabetes treatment plan, medication regimen, dosing schedule, or program and deliver one or more units of medication to the subject based on the diabetes treatment plan, medication regimen, dosing schedule, or program. In some embodiments, the diabetes treatment plan, medication regimen, dosing schedule, or program is pre-set, and the wearable device delivers to the subject medication based on the pre-set diabetes treatment plan, medication regimen, dosing schedule, or program. That is, there is no adjustment of the diabetes treatment plan, medication regimen, dosing schedule, or program based on, for example, user food intake or activity as described above.

In some embodiments, the diabetes treatment plan, medication regimen, dosing schedule, or program is dynamic. That is, there is adjustment of the diabetes treatment plan, medication regimen, dosing schedule, or program based on, for example, user food intake or activity as described herein.

In some embodiments, the controller can adjust the diabetes treatment plan, medication regimen, dosing schedule, or program based on a user's activity. For example, the diabetes treatment plan, medication regimen, dosing schedule, or program can be adjusted based on the detected heart rate, body temperature, estimated caloric expenditure, activity type, activity duration, or other parameter. In general, the diabetes treatment plan, medication regimen, dosing schedule, or program can be adjusted in any suitable way. For example, the controller can cause the medication delivery portion to deliver an additional dose, skip a dose, deliver a larger or smaller dose, postpone or expedite a scheduled dose, or otherwise change a medication delivery parameter or schedule in response to the user's activity, such as performing exercise or having a meal.

In some embodiments, the instruction, dose, or other medication delivery parameter or an adjustment to the diabetes treatment plan, medication regimen, dosing schedule, or program can be based on a detected user activity that may influence the glucose levels or glycemic response or insulin need of the user. In some embodiments, the activity that may influence the glucose levels or glycemic response or insulin need of the user or the details or parameters thereof (e.g., food amount and type, exercise duration, exercise type, etc.) can be input by the subject (user). Such input may be provided to the user input device of the wearable device, the portable electronic device, or some other device. In some embodiments, such an activity or the details or parameters thereof is automatically detected by the diabetes management system. For example, the automatic detection can use glucose concentration values and/or plasma insulin concentration estimations as inputs to detect an ongoing glycemic response to an activity in real time.

Automatic activity detection may be performed using a machine learning model to generate one or more output values based on one or more input variables. A machine learning model may be trained before it is used to make an inference from new input data. Training a machine learning model may involve, for example, determining parameters of the machine learning model, such as values of weights associated with one or more nodes of a neural network model. In some embodiments, a machine learning model may be trained in a supervised manner using a training data set that includes labeled training data. The labeled training data may include inputs and corresponding annotated outputs that the machine learning model is to approximate using learned weight values. In some other embodiments, a machine learning model may be trained in an unsupervised manner in which parameters of the machine learning model may be determined without using labeled training data.

In some embodiments, the parameter(s) of a machine learning model may be trained via loss minimization by feeding a training dataset to the machine learning model. For example, training a machine learning model may include optimization of parameters (e.g., weights or biases) of the machine learning model using techniques such as gradient descent and backpropagation techniques. A validation dataset may also be allocated (e.g., about 20%) from the training dataset to validate a trained machine learning model before deploying the machine learning model.

As used herein, a “classifier” may refer to a type of machine learning model that is trained to categorize inputs into one or more classes of a set of classes. Inputting data into a trained classifier may result in an output that categorizes the input data into an activity that affects glycemic index or insulin need or not an activity that affects glycemic index or insulin need. Thus, a properly trained classifier may provide an estimation of a mapping between input variables and discrete (as opposed to continuous) output variables. A classifier may be trained, for example, through a supervised fashion (including optimization and backpropagation), where the training data may comprise known and labeled information. In some cases, the trained classifier may use a regression technique such as logistic regression, which uses a logistic function to model a variable (such as a weighted sum of glucose concentration values and plasma insulin concentration (Ip) estimations) that may have multiple possible discrete outcomes, e.g., a binary or dichotomous outcome such as “activity that affects glycemic index or insulin need” or “not an activity that affects glycemic index or insulin need.” Logistic regression can map the predicted values to probabilities using, for example, a sigmoid function. A sigmoid function can map values between one end to another (e.g., 0 to 1), where one end may correspond to a meal and the other end may correspond to not a meal. In some implementations, one or more threshold values may be needed for the classifier to map the input data to one of the discrete outcomes. For example, if the probability is determined to be above a 0.2 threshold needed to be classified as a “0 ” but below a 0.8 threshold needed to be classified as a “1,” then the machine learning model may consider the determination invalid or null rather than one of the binary outcomes.

In some cases, the trained classifier may use a neural network such as a long short-term memory (LSTM) network, which is a type of recurrent neural network (RNN) capable of learning context and order. A RNN may perform the same task (looping back) for each successive element of a sequence, e.g., time series data, and the output may depend on the previous calculation. Each element may be associated with a corresponding layer of the RNN. The RNN may store an internal state, akin to a memory, which is determined based on the previously performed task at a previous time step and the current input at a current time step. Unlike a typical RNN, a LSTM may pass additional information (sometimes known as “cell memory”) from one time step to another, which helps capture long-term dependencies through multiple layers, carrying information between points in time and passing new information between states, which is more difficult to do with typical RNNs. That is, LSTMs can better remember previous information and use it for processing the current input. LSTMs can also discard irrelevant information. The output may be based on a sigmoid function (e.g., SoftMax), making LSTMs applicable to binary determinations such as “activity that affects glycemic index or insulin need” or “not an activity that affects glycemic index or insulin need.”

In some embodiments, the controller may be further configured to evaluate the different data provided by the sensors to further determine parameters of the activity. Such parameters may be useful in determining a total medication (e.g., insulin) need or an impact on a total medication (e.g., insulin) need of the subject based on the activity. For example, the controller can be configured to determine parameters such as estimates of a number of calories burned, mean heart rate, duration of activity, number of steps and the like. For example, the controller may be configured to determine based on the data received from the various sensors that the subject (user) participated in an exercise that included running for a duration of X minutes, and expending calories estimated to be C, where X and C are time and caloric values, respectively).

The parameters of the activity may be determined using machine learning techniques. In an example, machine learning classifiers may be trained to classify signals received from various sensors (e.g., accelerometer and/or gyroscope signals) to determine the parameters of the activity such as type (e.g., at rest, walking, lying down, running, climbing stairs, aerobic machines such as an elliptical machine, a rowing machine or a treadmill, duration, intensity, etc. For example, the controller may determine the duration of activity and the heart rate of the wearer, which may be used in conjunction to determine a total impact of the activity on the total medication (insulin) need. The parameters of the activity (e.g., sleeping, exercise) are relevant to the controller because such activity has an impact on the total glucose need and/or medication (insulin) need of the subject (user). The liver produces less glucose when a person is participating in aerobic activity than when a person is participating in anaerobic activity.

In some embodiments, the controller may determine and/or implement a response to the detected glucose level and/or the detection of an activity as described above without the aid of non-sensor devices (i.e. the controller alone receives various data from the various sensors, detects the glucose level and/or activity, determines, if applicable, the parameters of the activity with respect to an impact on an insulin need of the subject, and provides an appropriate instruction to the medication pump without another device). In some embodiments, the controller provides an instruction based on the glucose level. In some embodiments, the controller provides an instruction based on a diabetes treatment plan or other drug delivery regimen as described above. Such a diabetes treatment plan or medication regimen may be stored in a suitable memory location, such as in a memory included in the wearable device, the portable electronic device, or another device. In some embodiments, the controller may automatically modify delivery of the medication and calculate a modified dosage schedule for the modified dosage or dosages. The current amount of medication in the reservoir may also be factored in when recommending and/or implementing modifications to future medication delivery as described below.

In some embodiments, the controller may implement (and/or provide functionality for) a medication delivery algorithm or application (MDA). An example of an MDA may be an artificial pancreas (AP) application that may be configured to govern or control automated delivery of a drug or medication, such as insulin, to a user (e.g., to maintain euglycemia—a normal level of glucose in the blood). The MDA may, for example, receive information from additional applications or algorithms that execute on the controller or another component of the diabetes management system (e.g., the portable electronic device). For example, the controller may implement (and/or provide functionality for) other programs, such as an exercise detection and response algorithm as described above.

In some embodiments, the controller is configured to track information related to the medication that is contained within the medication reservoir. Such information can be or include identity information such as an active ingredient (e.g., insulin), the dosage of the medication, the brand or manufacturer of the medication, or any other similar property or factor of the medication. Such information can be entered by a user, transmitted from another device, or automatically retrieved by the controller from another device. For example, when a user fills or replaces the reservoir, that user can manually enter the information related to the medication. In another example, the user can image or scan the medication, a replacement reservoir, a prescription, a pharmacy information package, or some other similar article using a scanner or camera included in the portable electronic device or some other device. The imaging or scanning can input the information directly, for example through use of a barcode, 2D barcode, or other similar visual information encoding scheme. The imaging or scanning can initiate an information retrieval process in which the information is retrieved from another device. For example, the portable electronic device or some other device can image or scan an article that encodes some identifier of the medication such that the controller can retrieve the necessary information about the medication from a remote database (e.g., a manufacturer database). The remote database can be accessed, for example, via some connection to a remove device via the internet. In another example, the user can input, access, or retrieve the information related to the medication using their smartphone or other electronic device, which then transmits the identity or parameter of the medication to the controller. For example, prescription information can be accessed on the user's smartphone then transmitted to the controller via a connection between the controller and the smartphone. Such a connection can be provided by a wired connection or a wireless connection via a suitable wireless communication protocol (e.g., Wi-Fi, Bluetooth, etc.).

In some embodiments, the controller is configured to track information related to the delivery of the medication that is contained within the medication reservoir. For example, the controller can track a medication history of the user. Such a medication history can include any suitable information related to past use or administration of the medication. For example, the medication history can include a total amount of medication administered in a given time period. Such information can be useful to prevent overdosing of the medication (e.g., too much insulin being administer and causing adverse events). The medication history can also be used by the controller to correlate medication delivery with a user response, such as the patient's blood sugar response to a certain size dosage of insulin.

In some embodiments, the controller is configured to track information related to the user data collected by the sensor portion. For example, the controller can track an exercise history, a user glucose history, a user heart rate activity, a user temperature activity, and the like. These user data histories can be useful in determining parameters of the diabetes treatment or management plan, the delivery of the medication, user compliance with behavior instructions, and combinations of these. For example, user blood glucose data present in a user blood glucose history can be correlated with medication delivery (e.g. present in a medication delivery history) to determine an estimated user response to a given amount of medication being delivered. User exercise data present in an exercise history can be correlated with the user blood glucose history to determine an estimated impact on blood glucose level based on exercise the user performs. In this way, the system can tailor aspects of the diabetes treatment or management to the specific parameters of the user. Such tailoring can be made automatically as the user continues use of the system.

In some embodiments, the controller is configured to track information related to the behavior that is tracked with the behavior tracking device. For example, the controller can include a sleep history when the behavior tracking device is configured to track a user's sleep. When the behavior tracking device is configured to track a user's non-insulin medication use or compliance, the controller can track a user's history of such use or compliance. The controller can correlate such a behavioral history with one of the histories described above. Such correlation can be useful for tailoring aspects of the diabetes treatment or management to the specific parameters of the user. Such tailoring can be made automatically as the user continues use of the system. For example, the system can automatically adjust an amount of medication that should be or must be provided to the user to maintain, for example, a certain blood glucose level based on the system detecting a user performing a behavior tracked by the behavior tracking device. When the behavior tracking device detects a user taking a certain non-insulin medication that is associated with an increased insulin need, the system can adjust to automatically provide additional insulin to the user. Such additional insulin can be provided in increased or additional dosages. The amount of the additional insulin can be based on a history of increased insulin need correlated with the behavior tracking device detecting that behavior.

In some embodiments, the controller is configured to provide an alert to a user. In general, the alert can be any suitable notification. Such a notification can bring to the user's attention some information related to the diabetes treatment plan, medication regimen, dosing schedule, program, device, system, behavior, or combination of these. In some embodiments, a notification can require user input or be related to a task or activity that requires user input. For example, a notification can be provided to remind a user that it is time to take one or more units of medication and/or non-insulin medication. Such a notification can be automatically provided by the controller based on the diabetes treatment plan, medication regimen, dosing schedule, or program. In another example, a notification can be provided to remind a user to refill their prescription. In another example, a notification can be provided to alert the user to a low level of a medication remaining in the reservoir (e.g., a “low medication alert”). In some embodiments, the controller can track a quantity of units medication held in the reservoir. Such a quantity can be used to determine when to provide the low medication alert. In some embodiments, the quantity can be tracked based on an input quantity provided to the controller during filling and updated when a unit of the medication (e.g., insulin) is delivered. When the quantity reaches a threshold value, the low medication alert can be provided. In another example, a notification can be provided to the user to engage in or abstain from some behavior. Such a behavior can be associated with the behavior tracking device. For example, the user can be reminded to abstain from sweets or other foods that have negative impacts on diabetes management. The user can be reminded to choose better foods associated with positive impacts on diabetes management or which may help or result in the user losing weight. The user can be reminded to take a non-insulin medication. In this way, the diabetes treatment plan can include a behavioral modification component.

In some embodiments, the notification can be generated by the controller. In some embodiments, the notification can be provided to the user on a notification device which is integral with the wearable device. In some embodiments, the notification can be provided to the user on the portable electronic device. For example, the user's smartphone can be wirelessly connected to the wearable device such that a notification can be provided to the user via the smartphone. In some embodiments, a notification can be provided even when the user's portable electronic device (e.g., smartphone) is not currently connected to the wearable device and/or the behavior tracking device. For example, a connection between the smartphone and the wearable device can transfer information related to future notifications to the user's smartphone (e.g., future medication reminders based on the regimen, reminders to exercise, reminders to abstain from sweets, etc.). Such pre-loaded notifications can be configured to be displayed by the smartphone under appropriate conditions, such as at a certain time.

### Portable Electronic Device

The portable electronic device is configured to communicate wirelessly to the wearable device, the behavior tracking device, or some other component of the diabetes management system. In general, the portable electronic device can be any suitable such device, such as a smartphone, tablet, computer, smartwatch, or similar such device capable of receiving a wireless transmission from the diabetes management system or a component thereof. Such a transmission can be provided by a wireless connection via a suitable wireless communication protocol (e.g., Wi-Fi, Bluetooth, etc.).

In general, the portable electronic device can serve multiple functions related to the diabetes management system and/or the use thereof.

For example, a user's smartphone can be wirelessly connected to the diabetes management system or a component thereof such that a notification can be provided to the user via the smartphone. In some embodiments, a notification can be provided even when the user's electronic device (e.g., smartphone) is not currently connected to the diabetes management system or a component thereof. For example, a connection between the smartphone and the diabetes management system or a component thereof can transfer information related to future notifications to the user's smartphone (e.g., future medication reminders or future behavior reminders). Pre-loaded notifications can be configured to be displayed by the smartphone under appropriate conditions, such as at a certain time.

In some embodiments, the diabetes management system or a component thereof is controlled by a suitable application or piece of software configured to run on the portable electronic device. For example, the portable electronic device can be a smartphone and the software can be a smartphone app. Such a piece of software or app can control the transfer of information between the portable electronic device and the diabetes management system or a component thereof. Such a piece of software can provide the user a convenient interface for controlling the diabetes management system or a component thereof using the portable electronic device. For example, a smartphone running a smartphone app can store the diabetes treatment plan, medication regimen, dosing schedule, or program. In another example, the smartphone app can track a user's non-insulin medication or behavior compliance. The user can indicate to the smartphone app that they have taken the dose. The smartphone app can store the user's medication history (e.g., what medications have been taken and when) and/or behavioral history. The medication history and/or behavioral history can be transmitted to diabetes management system or component thereof (e.g., controller), which can update relevant information stored therein. That behavioral history can then be used, for example, in the behavior adjustment component of the diabetes treatment plan.

The user's portable electronic device can serve as a user input device or interface to allow the user to control, adjust, or provide instructions to the diabetes management system or a component thereof. For example, the keyboard or touchscreen of the portable electronic device can be used as a convenient interface for inputting information into the diabetes management system or a component thereof. Such information can be, for example, the identity or other information related to the medication in the medication reservoir. For example, the user can manually enter such information to the controller via a touch screen, manually enter such information into their smartphone and transfer to the controller via some wireless communication protocol (e.g., Wi-Fi, Bluetooth, etc.). The user can automatically retrieve the information related to the medication in the medication reservoir, for example, by scanning and/or retrieving such information as described above. In some embodiments, the user can manually input or have the portable electronic device automatically transmit information related to the behavior associated with the behavior tracking device.

The user's portable electronic device can serve as a system output device or interface to allow the user to view, access, or analyze the parameters or functions of the diabetes management system or a component thereof. For example, the portable electronic device can serve to display or present a notification described above to the user. In some embodiments, the portable electronic device is configured to provide a user notification based on the user behavior associated with the medication delivered by the system. In some embodiments, the portable electronic device can serve to display to the user the diabetes treatment plan, medication regimen, dosing schedule, behavior, or program.

The diabetes management system can also be configured to, for example, deliver a dose of medication based on a user input. For example, a user can provide an instruction to the wearable device or control to deliver or provide to the user a dose of insulin. This may be referred to as “manual” or “user-initiated” delivering.

### Method

The present application also relates to a method of managing diabetes in a subject. In some embodiments, the method involves detecting a glucose level of the subject using the sensor portion of the diabetes management system and delivering to the subject a dose of a medication based on the glucose level, the delivering being performed by the diabetes management system.

In some embodiments, the method further includes detecting a user behavior associated with the medication delivered by the diabetes management system, wherein the dose of the medication is based on the user behavior. By detecting the user behavior associated with the medication, the effects of the that behavior on the diabetes treatment or management plan can be accounted for, as described above. For example, exercise can have dramatic impacts on the user's insulin needs both during the exercise and for hours after the exercise. The consumption of certain types of foods can also have dramatic impacts on the user's insulin needs.

In some embodiments, the method further includes detecting a user behavior that is not associated with the medication delivered by the diabetes management system but which has an immediate effect on the dose of the medication. By detecting the user behavior not associated with the medication, the effects of the that behavior on the diabetes treatment or management plan can be accounted for, as described above. For example, the user can take a medication that is intended to treat a different disease or acute condition (e.g., a painkiller or cardiovascular medication) that can have dramatic impacts on the user's insulin needs immediately after taking the medication.

In some embodiments, the method further includes detecting a user behavior that is not associated with the medication delivered by the diabetes management system and which does not have an immediate effect on the dose of the medication. By detecting the user behavior not associated with the medication, the diabetes treatment or management plan can incorporate such behavior. For example, if the method can detect long term patterns of exercise or can detect smoking, such activities can be accounted for on a long-term basis even if they do not have an immediate impact on the user's insulin needs.

Such behavior detection can be associated with the providing of information related to the behavior and/or the diabetes treatment or management to the user. For example, the user can be provided with notifications as described above. Such notifications can be intended to encourage or discourage the user from engaging in the specific behavior. This type of notification can be used in a portion of a diabetes treatment or management plan that involves behavior modification. For example, the behavior modification can be intended to help the user lose weight, such as by choosing different foods, changing the amount of food the user is consuming, changing the frequency, intensity, or type of exercise, quitting smoking, ensuring the user continues to regularly take a medication associated with the diabetes treatment or management, ensuring the user continues to regularly take a medication not associated with the diabetes treatment or management, etc.

In some embodiments, the method further includes providing a user notification based on the user behavior associated with the medication delivered by the system. In some embodiments, the method further includes providing a user notification based on the user behavior not associated with the medication delivered by the system. The user notification can be provided by the portable electronic device of the diabetes management system as described above.

In some embodiments, the method involves providing to the user instructions to perform certain activities other than administering or using insulin as part of a diabetes management or treatment plan. For example, the diabetes management or treatment plan can include a weight loss program or regimen, a regimen of a non-insulin medication, a smoking cessation program, a program related to rest or sleep, a diet or nutrition program, and the like. The notifications described above can form a part of the instructions provided to the user. For example, the notifications can provide encouragement and/or discouragement for the user to perform certain activities as related to the diabetes management or treatment plan. Such encouragement and/or discouragement can be useful for helping the user form habits or continue to perform activities other than insulin administration or use that better manage the patient's diabetes and/or help improve the function of the diabetes management system.

In some embodiments, the medication is insulin as described above.

The examples below are intended to further illustrate protocols for constructing the system or its components and performing the method described herein and are not intended to limit the scope of the claims.

Where a numerical limit or range is stated herein, the endpoints are included. Also, all values and subranges within a numerical limit or range are specifically included as if explicitly written out.

### EXAMPLES

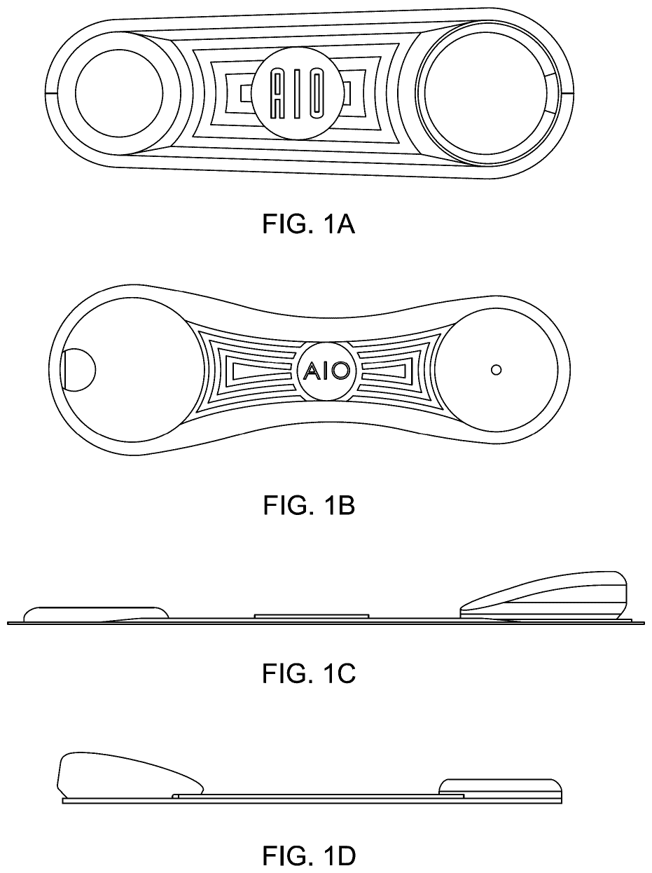

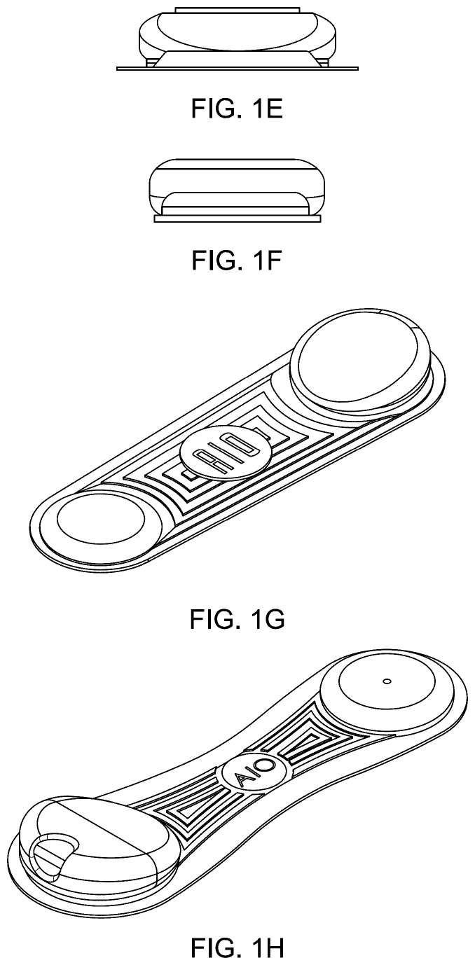

FIG. 1A shows a top-down schematic view of an exemplary embodiment of a wearable device according to the present specification. In FIG. 1A, the wearable device is shown with the sensor portion oriented on the left of the figure and the medication delivery portion oriented on the right of the figure. FIG. 1B shows a top-down schematic view of an exemplary embodiment of a wearable device according to the present specification. In FIG. 1B, the wearable device is shown with the sensor portion oriented on the right of the figure and the medication delivery portion oriented on the left of the figure, opposite of FIG. 1A.

FIG. 1C shows a side-on schematic view of an exemplary embodiment of a wearable device according to the present specification. In FIG. 1C, the wearable device is shown with the sensor portion oriented on the left of the figure and the medication delivery portion oriented on the right of the figure. FIG. 1D shows a side-on schematic view of an exemplary embodiment of a wearable device according to the present specification. In FIG. 1D, the wearable device is shown with the sensor portion oriented on the right of the figure and the medication delivery portion oriented on the left of the figure, opposite of FIG. 1C.

FIG. 1E shows a frontal schematic view of an exemplary embodiment of a wearable device according to the present specification. In FIG. 1E, the wearable device is shown with the medication delivery portion oriented toward the viewer. FIG. 1F shows a frontal schematic view of an exemplary embodiment of a wearable device according to the present specification. In FIG. 1F, the wearable device is shown with the sensor portion oriented toward the viewer, opposite of FIG. 1E.

FIG. 1G shows a three-quarters schematic view of an exemplary embodiment of a wearable device according to the present specification. In FIG. 1G, the wearable device is oriented with the sensor portion closer to the bottom of the figure and the medication delivery portion closer to the top of the figure. FIG. 1H shows a three-quarters schematic view of an exemplary embodiment of a wearable device according to the present specification. In FIG. 1H, the wearable device is oriented with the sensor portion closer to the top of the figure and the medication delivery portion closer to the bottom of the figure, opposite of FIG. 1G.

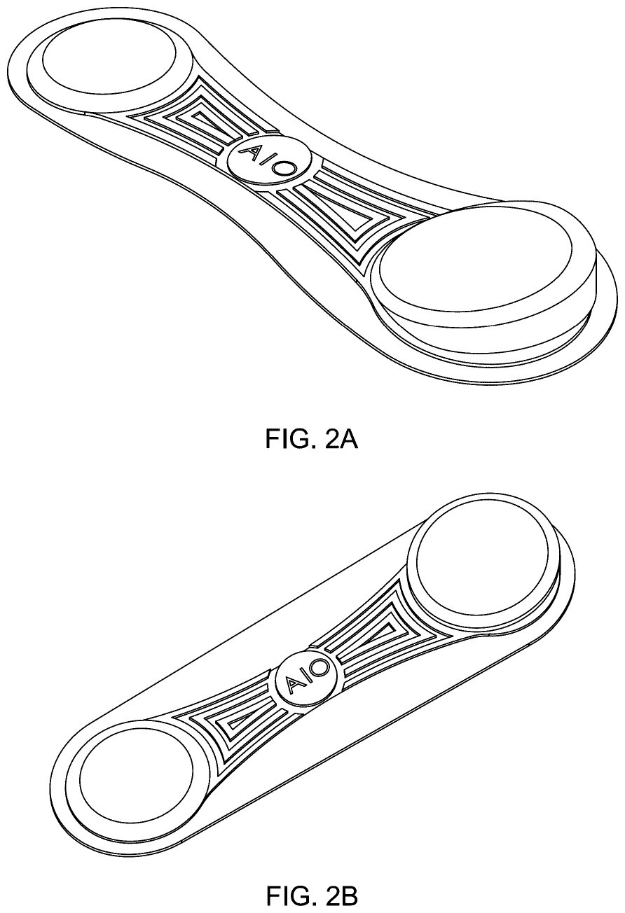

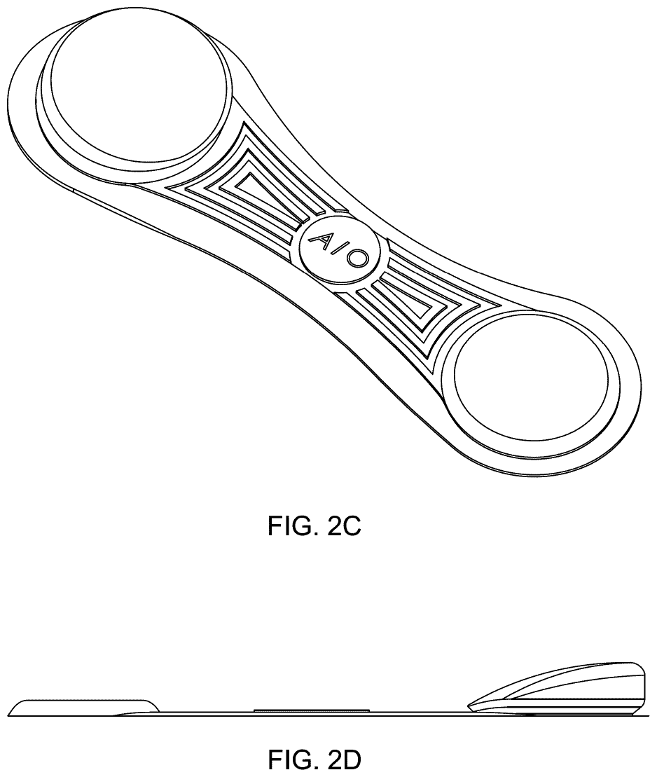

FIGS. 2A-2D show renderings of an exemplary embodiment of a wearable device according to the present specification with FIGS. 2A-2C showing different three-quarters views and FIG. 2D showing a side-on view.

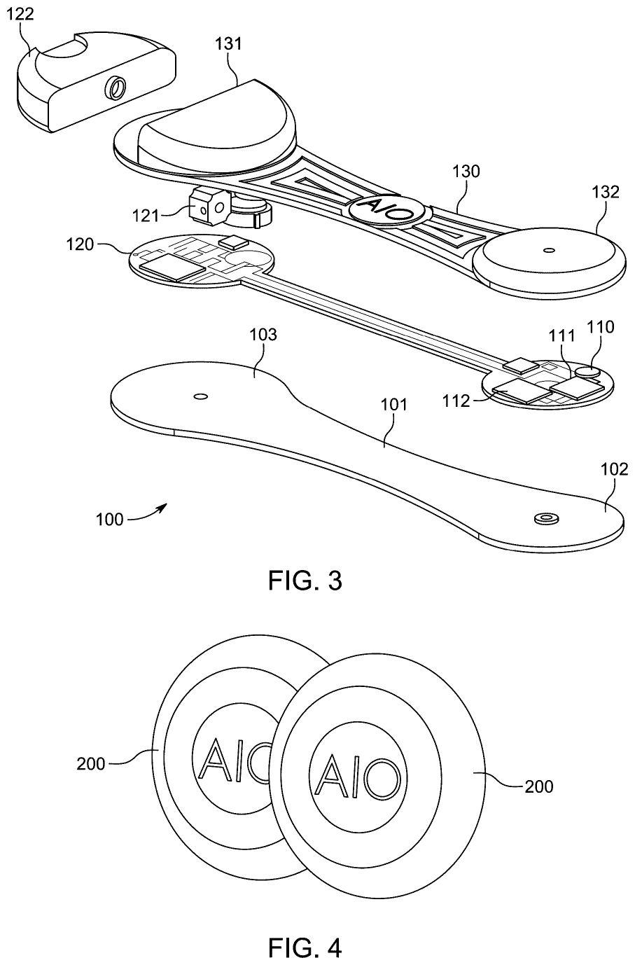

FIG. 3 shows an expanded view of an exemplary embodiment of a wearable device according to the present specification. The device 100 is shown with an exemplary elongated support patch 101, having a first rounded end 102 and a second rounded end 103. The first rounded end 102 is smaller than the second rounded end 103. The wearable device 100 is shown with the skin side of the elongated support patch 101, and therefore the adhesive disposed thereon, not visible.

Disposed on the first rounded end 102 of the exemplary elongated support patch 101 is a sensor portion 110. The exemplary sensor portion 110 includes an electrochemical glucose sensor 111 and a wireless communication module 112. Disposed on the second rounded end 103 of the exemplary elongated support patch 101 is a medication delivery portion 120. The medication delivery portion includes a medication pump 121 and a medication reservoir 122. Disposed on the wearable device 100 is a cover 130 that includes a sensor portion cover 132 and a medication delivery portion cover 131.

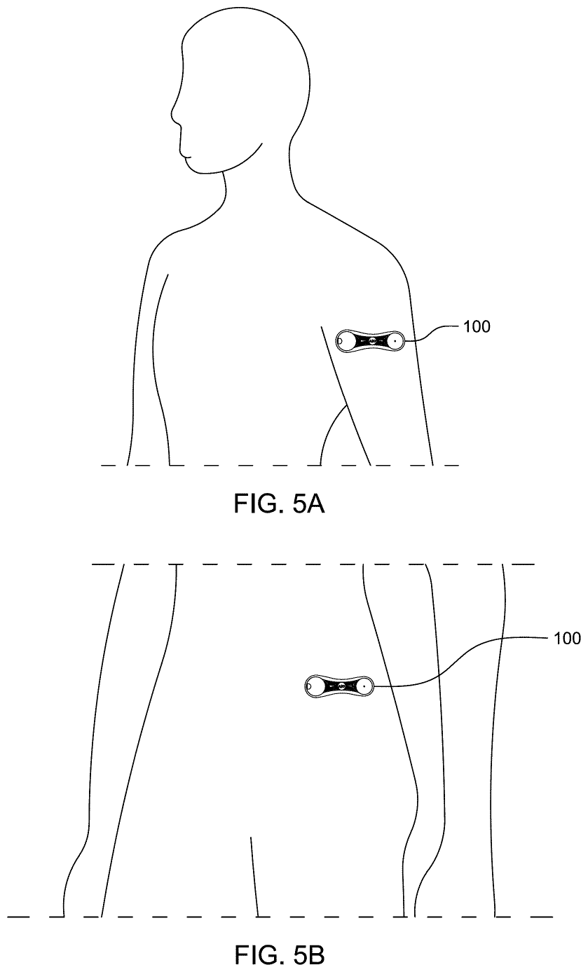

FIG. 4 shows a rendering showing an exemplary embodiment of a behavior tracking device 200 according to the present specification.

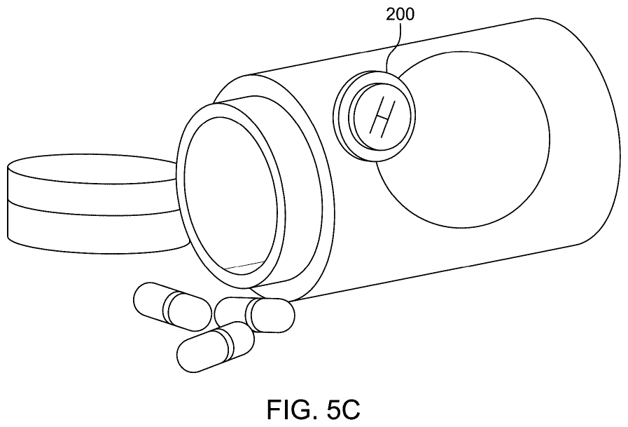

FIGS. 5A-5C show exemplary depictions of performing a method of diabetes management using the system according to the present specification. FIGS. 5A-5B show a subject wearing the wearable device 100. In FIG. 5A, a subject is shown wearing the wearable device 100 on their arm. In FIG. 5B, a subject is shown wearing the wearable device 100 on their lower abdomen. FIG. 5C showing the behavior tracking device 200 placed on a medication bottle.

Where a numerical limit or range is stated herein, the endpoints are included. Also, all values and subranges within a numerical limit or range are specifically included as if explicitly written out.

Numerous modifications and variations of the present invention are possible in light of the above teachings. It is therefore to be understood that, within the scope of the appended claims, the invention may be practiced otherwise than as specifically described herein.

1. A diabetes management system, comprising
a wearable device, comprising
an elongated support patch including an adhesive disposed on a skin side of the elongated support patch, wherein the elongated support patch has a first rounded end and a second rounded end, with the first rounded end smaller than the second rounded end,
a sensor portion disposed on a non-skin side of the elongated support patch at the first rounded end and comprising an electrochemical glucose sensor, and a wireless communication module, and
a medication delivery portion disposed on a non-skin side of the elongated support patch at the second rounded end and comprising a medication reservoir and a medication pump;
a behavior tracking device comprising a tracking wireless communication module;
a portable electronic device configured to communicate wirelessly to the wearable device and the behavior tracking device; and
a controller,
wherein the sensor portion is configured to detect a glucose level of a subject and the controller is configured to receive the glucose level and to transmit to the medication pump an instruction which causes the medication pump to deliver a dose to the subject of a medication based on the glucose level.

2. The diabetes management system of claim 1, wherein the elongated support patch is formed from a polymer film.

3. The diabetes management system of claim 1, wherein the elongated support patch is formed from a textile.

4. The diabetes management system of claim 1, wherein the elongated support patch has a maximum thickness of 0.25 to 5 mm.

5. The diabetes management system of claim 1, wherein
the sensor portion further comprises a heartbeat sensor and a temperature sensor,
the controller is further configured to receive a heartbeat level and a temperature of the subject, and
the instruction is further based on the heartbeat level and the temperature of the subject.

6. The diabetes management system of claim 1, wherein the sensor portion has a maximum thickness of 1 to 8 mm.

7. The diabetes management system of claim 1, wherein the medication delivery portion further comprises a microneedle.

8. The diabetes management system of claim 1, wherein the medication reservoir has a medication solution capacity of 1 to 5 mL.

9. The diabetes management system of claim 1, wherein the medication delivery portion has a wedge-shaped cross section having a maximum thickness of 5 to 15 mm.

10. The diabetes management system of claim 1, wherein the medication is insulin.

11. The diabetes management system of claim 1, wherein the controller is included in the sensor portion of the wearable device.

12. The diabetes management system of claim 1, wherein the controller is included in the portable electronic device.

13. The diabetes management system of claim 1, wherein the behavior tracking device is configured to track a user behavior associated with the medication delivered by the system.

14. The diabetes management system of claim 13, wherein the user behavior is associated with a change in a dosage of the medication delivered by the system.

15. The diabetes management system of claim 13, wherein the behavior tracking device is configured to transmit a behavior signal to the controller, the controller is configured to receive the behavior signal, and the instruction is further based on the behavior signal.

16. The diabetes management system of claim 13, wherein the portable electronic device is configured to provide a user notification based on the user behavior associated with the medication delivered by the system.

17. A method of managing diabetes in a subject, the method comprising
detecting a glucose level of the subject using the sensor portion of the diabetes management system of claim 1; and
delivering to the subject a dose of a medication based on the glucose level, the delivering being performed by the diabetes management system of claim 1.

18. The method of claim 17, further comprising
detecting a user behavior associated with the medication delivered by the diabetes management system of claim 1,
wherein the dose of the medication is based on the user behavior.

19. The method of claim 18, further comprising providing a user notification based on the user behavior associated with the medication delivered by the system, the user notification being provided by the portable electronic device of the diabetes management system of claim 1.

20. The method of claim 17, wherein the medication is insulin.
